(19) United States
(12) Patent Application Publication
(10) Pub. No.: US 2018/0099917 A1
(43) Pub. Date: Apr. 12, 2018

(54) REVERSIBLY REDUCIBLE MATERIALS AND USE THEREOF

(71) Applicant: University of Kentucky Research Foundation, Lexington, KY (US)

(72) Inventors: John Anthony, Lexington, KY (US); Susan A. Odom, Lexington, KY (US)

(21) Appl. No.: 15/730,428

(22) Filed: Oct. 11, 2017

Related U.S. Application Data
(60) Provisional application No. 62/406,733, filed on Oct. 11, 2016.

(51) Int. Cl. C07C 50/12; C07C 211/57; H01M 8/18; C07C 211/42

(52) U.S. Cl. CPC C07C 50/12; C07C 211/57; H01M 2300/0025; C07C 211/42; H01M 8/188

(57) ABSTRACT

Provided herein are a reversibly reducible material and a method of forming a reversibly reducible material. The reversibly reducible material includes the molecular formula:

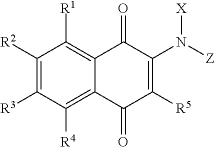

wherein each of R¹, R², R³, and R⁴ are independently selected from the group consisting of hydrogen, oxygen, alkyl, cycloalkyl, O-alkyl, amine, quaternary ammonium, and sulfonate; R⁵ is selected from the group consisting of hydrogen, halogen, alkyl, alkoxy, and amine; X is selected from the group consisting of hydrogen, branched or un-branched alkyl chain having 1-8 atoms containing 0-3 oxygen or nitrogen atoms, and substituted or unsubstituted aryl; and Z is selected from the group consisting of branched or un-branched alkyl chain having 1-8 atoms containing 0-3 oxygen or nitrogen atoms, and substituted or unsubstituted aryl. The method of forming a reversibly reducible material comprising reacting a quinone with an amine in an ethereal solvent. Also provided herein is a negolyte.

### RELATED APPLICATIONS

This application claims the benefit of U.S. Provisional Application Ser. No. 62/406,733, filed Oct. 11, 2016, the entire disclosure of which is incorporated herein by this reference.

### TECHNICAL FIELD

The presently-disclosed subject matter generally relates to highly soluble, stable, reversibly reducible materials and methods of use thereof. In particular, certain embodiments of the presently-disclosed subject matter relate to negolytes for use in redox-flow batteries and methods of forming such negolytes.

### BACKGROUND

Redox flow batteries (RFBs), which function by storing electrochemical energy in liquid electrolyte formulations, are promising technologies for energy storage on medium to large scales. Since the energy is stored in a liquid form, increasing the capacity of RFBs is largely a matter of increasing the size of active-material storage tanks, or increasing the concentration of the active component (which requires improved solubility in the electrolyte solution). While existing RFBs based upon aqueous vanadium chemistry are robust and long-lived, they rely upon expensive transition metals as active materials that form redox active species. Additionally, these RFBs are aqueous-based and, therefore, are limited by the electrochemical stability window of water (about 1.5 V).

One possibility for accessing higher voltages includes the use of non-aqueous electrolytes, which are generally not compatible with transition metal-based active materials. Although such systems have the potential for increasing the voltage of the system and reducing cost by exchanging scarce transition metals for plentiful carbon-based materials, the development and commercialization of organic non-aqueous redox flow batteries is largely in its infancy due to challenges in long term stability, atom economy, and solubility/energy density.

Accordingly, there exists a need for stable electroactive materials having sufficient atom economy, electrochemical reversibility, and solubility for use in redox flow batteries.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments, the presently-disclosed subject matter includes a reversibly reducible material comprising the molecular formula:

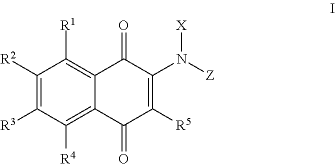

wherein each of R¹, R², R³, and R⁴ are independently selected from the group consisting of hydrogen, oxygen, alkyl, cycloalkyl, O-alkyl, amine, quaternary ammonium, and sulfonate; R⁵ is selected from the group consisting of hydrogen, halogen, alkyl, alkoxy, and amine; X is selected from the group consisting of hydrogen, branched or un-branched alkyl chain having 1-8 atoms containing 0-3 oxygen or nitrogen atoms, and substituted or unsubstituted aryl; and Z is selected from the group consisting of branched or un-branched alkyl chain having 1-8 atoms containing 0-3 oxygen or nitrogen atoms, and substituted or unsubstituted aryl.

In some embodiments, X and Z are connected by an alkyl or heteroalkyl chain containing O, S, or N. In some embodiments, the X, Z, and the N to which they are bound form a common aromatic heterocycle selected from the group consisting of a carbazole, phenothiazine, pyrrole, and/or imidazole. Additionally or alternatively, in some embodiments, R¹, R², R³, and R⁴ comprise an alkyl, O-alkyl, or amine system. In one embodiment, at least one of R¹ and R², R² and R³, and R³ and R⁴ are linked together to form a ring structure consisting of 3-6 atoms. In another embodiment, the molecular formula includes one or more of the following compounds:

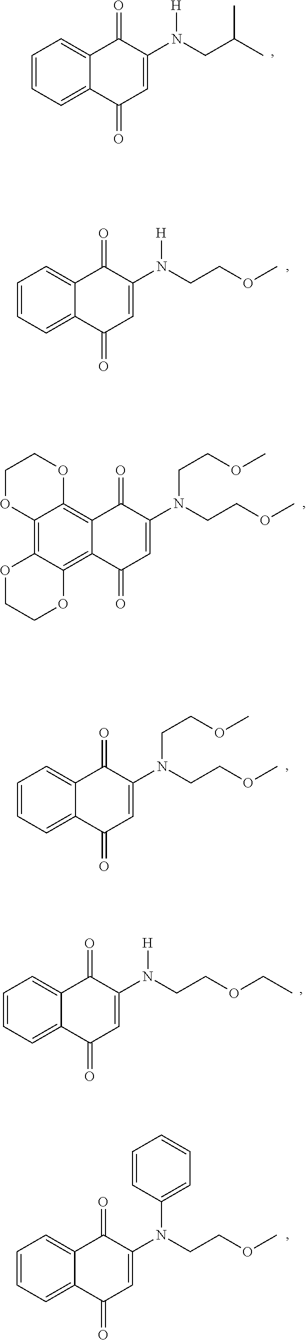 [SMILES: CC(C)CNC1=CC(=O)c2ccccc2C1=O ; COCCNC1=CC(=O)c2ccccc2C1=O ; COCCN(CCOC)C1=CC(=O)c2c3c(c4c(c2C1=O)OCCO4)OCCO3 ; COCCN(CCOC)C1=CC(=O)c2ccccc2C1=O ; CCOCCNC1=CC(=O)c2ccccc2C1=O ; COCCN(C1=CC(=O)c2ccccc2C1=O)c1ccccc1]

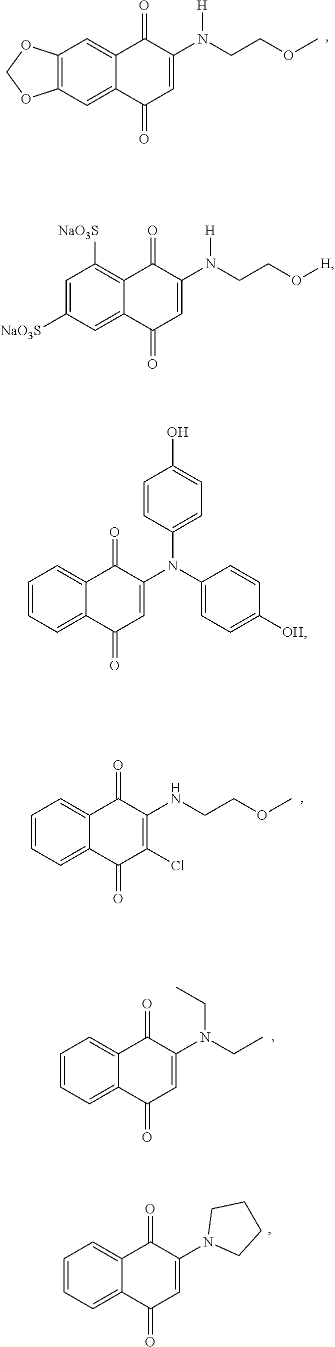

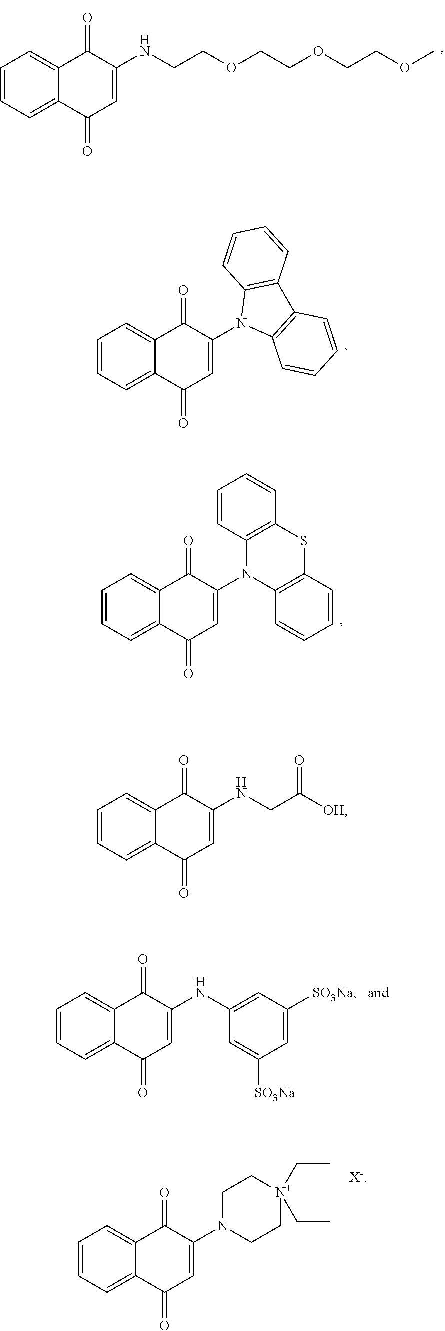

Also provided herein, in some embodiments, is a method of forming a reversibly reducible material comprising reacting a quinone with an amine in an ethereal solvent. In one embodiment, the method comprises reacting a 1,4-naphthoquinone and the amine in a tetrahydrofuran solvent according to the following reaction:

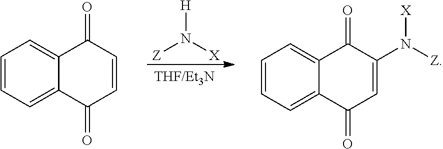

In another embodiment, the ethereal solvent is selected from the group consisting of dioxane, diethyl ether, glyme, and combinations thereof. In a further embodiment, the reaction includes a three-fold excess of amine in place of the triethylamine or Hünig's base.

Further provided herein, in some embodiments, is a negolyte comprising a reversibly reducible material and a redox flow battery solvent. In some embodiments, the reversibly reducible material is arranged and disposed to reversibly accept up to two electrons. In some embodiments, the reversibly reducible material comprises the molecular formula:

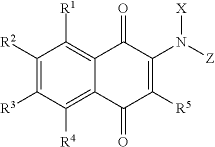

wherein each of R¹, R², R³, and R⁴ are independently selected from the group consisting of hydrogen, oxygen, alkyl, cycloalkyl, O-alkyl, amine, quaternary ammonium, and sulfonate; R⁵ is selected from the group consisting of hydrogen, halogen, alkyl, alkoxy, and amine; X is selected from the group consisting of hydrogen, branched or un-branched alkyl chain having 1-8 atoms containing 0-3 oxygen or nitrogen atoms, and substituted or unsubstituted aryl; and Z is selected from the group consisting of branched or un-branched alkyl chain having 1-8 atoms containing 0-3 oxygen or nitrogen atoms, and substituted or unsubstituted aryl.

In some embodiments, X and Z are connected by an alkyl or heteroalkyl chain containing O, S, or N. In some embodiments, the X, Z, and the N to which they are bound form a common aromatic heterocycle selected from the group consisting of a carbazole, phenothiazine, pyrrole, and/or imidazole. In some embodiments, R¹, R², R³, and R⁴ comprise an alkyl, O-alkyl, or amine system. In one embodiment, at least one of R¹ and R², R² and R³, and R³ and R⁴ are linked together to form a ring structure consisting of 3-6 atoms. In another embodiment, the molecular formula includes one or more of the following compounds:

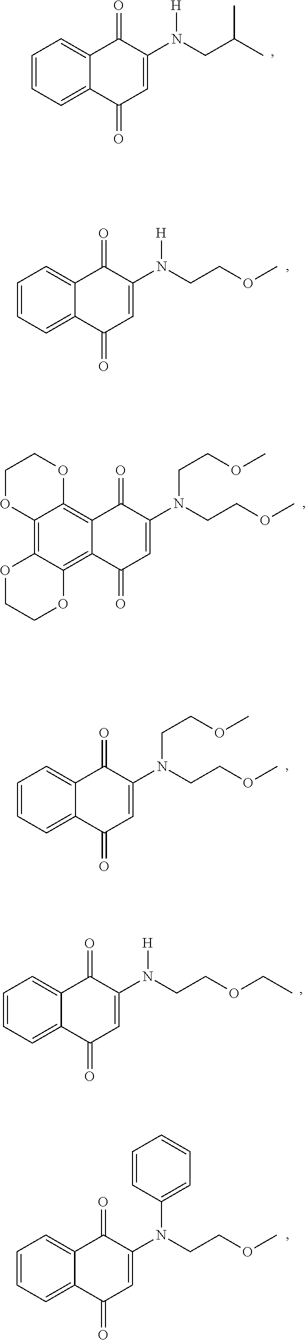 [SMILES: CC(C)CNC1=CC(=O)c2ccccc2C1=O ; COCCNC1=CC(=O)c2ccccc2C1=O ; COCCN(CCOC)C1=CC(=O)c2c3c(c4c(c2C1=O)OCCO4)OCCO3 ; COCCN(CCOC)C1=CC(=O)c2ccccc2C1=O ; CCOCCNC1=CC(=O)c2ccccc2C1=O ; COCCN(C1=CC(=O)c2ccccc2C1=O)c1ccccc1]

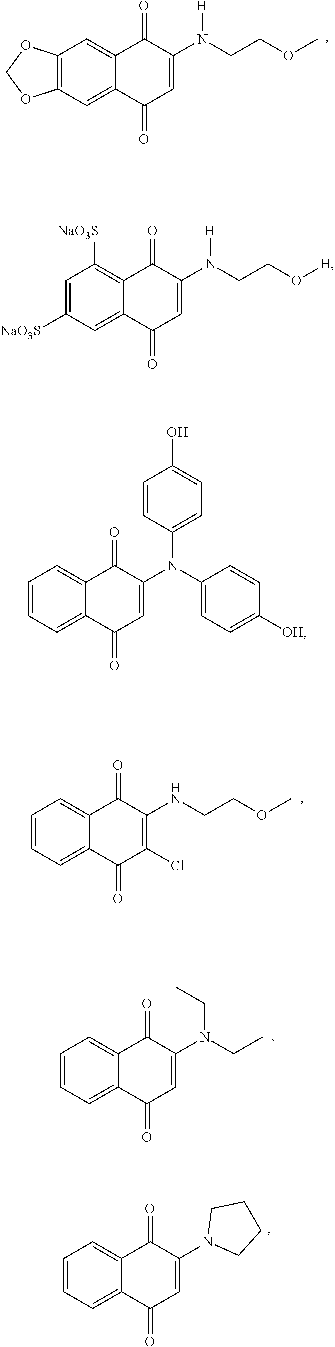

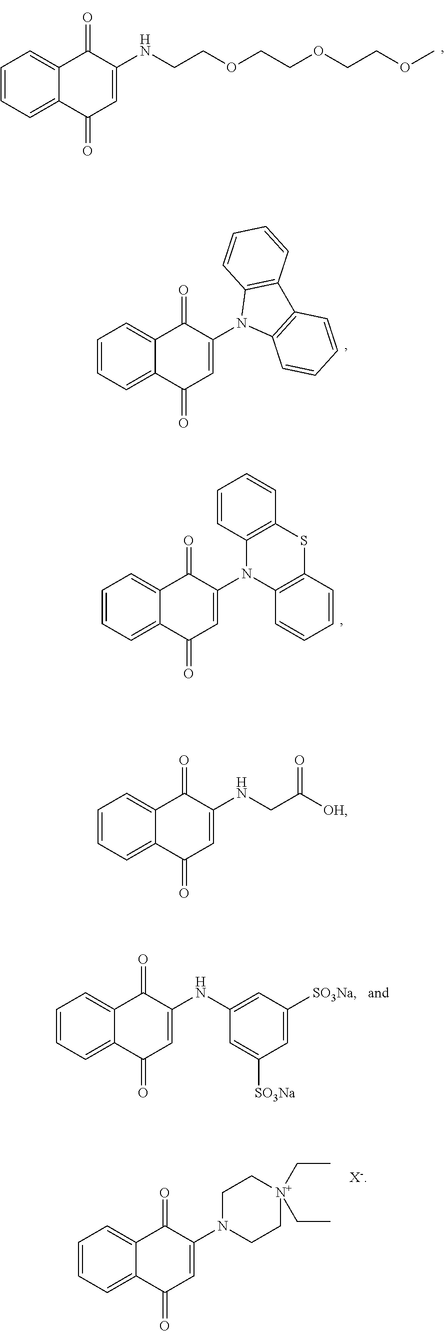

In some embodiments, the solvent is selected from the group consisting of acetonitrile, propylene glycol, propylene carbonate, dimethyl acetamide, and dimethyl formamide.

Further features and advantages of the presently-disclosed subject matter will become evident to those of ordinary skill in the art after a study of the description, figures, and non-limiting examples in this document.

### BRIEF DESCRIPTION OF THE DRAWINGS

FIG. 1 shows a graph illustrating solubility and electrochemical reversibility of a methoxyethyl derivative in acetonitrile, according to an embodiment of the disclosure.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter. Unless defined otherwise, all technical and scientific terms used herein have the same meaning as is commonly understood by one of skill in the art to which the invention(s) belong.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter includes highly soluble, stable, reversibly reducible materials, methods of forming such materials, and methods of using such materials. The reversibly reducible materials include materials capable of accepting at least one electron to form a reduced state while having and/or maintaining long-term stability in both the neutral and all reduced states. As used herein, the term “long-term stability” refers to materials that show no decomposition or side reactions over the course of several months.

In some embodiments, the reversibly reducible materials are capable and/or prefer double reduction (i.e., acceptance of two electrons). In one embodiment, the reversibly reducible material includes a plain or functionalized 1,4-naphthoquinone core. In another embodiment, the 1,4-naphthoquinone core provides high stability in the quinone state, and enhanced stability in the fully aromatic doubly-reduced stated (hydroquinone). In a further embodiment, the 1,4-naphthoquinone core is functionalized at the 2 position with amine groups to manipulate the reduction potential of the quinone in order to increase cell voltage. For example, in certain embodiments, the molecular form of the reversibly reducible material follows the formula:

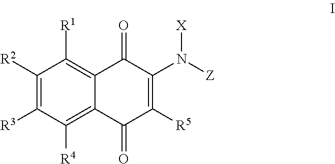

where each of R¹, R², R³, and R⁴ independently includes, but is not limited to, a hydrogen, oxygen, alkyl, cycloalkyl, O-alkyl, amine, quaternary ammonium, and/or sulfonate moiety; R⁵ includes, but is not limited to, a hydrogen, halogen, alkyl, alkoxy, and/or amine moiety; X includes, but is not limited to, a hydrogen, branched or un-branched alkyl chain having 1-8 atoms containing 0-3 oxygen or nitrogen atoms, and/or substituted or unsubstituted aryl moiety; and Z includes, but is not limited to, a branched or un-branched alkyl chain having 1-8 atoms containing 0-3 oxygen or nitrogen atoms, and/or substituted or unsubstituted aryl moiety.

In some embodiments, X and Z are connected by an alkyl or heteroalkyl chain containing O, S, or N. In one embodiment, the N group in such a ring, when present, is secondary, tertiary, or quaternary. In another embodiment, the nitrogen moiety may be part of a common aromatic heterocycle, such as, but not limited to, a carbazole, phenothiazine, pyrrole, and/or imidazole. Additionally or alternatively, in some embodiments, R¹, R², R³, and R⁴ comprise an alkyl, O-alkyl, or amine system. In one embodiment, at least one of R¹ and R², R² and R³, and R³ and R⁴ are linked together to form a ring structure consisting of 3-6 atoms. In another embodiment, the molecular formula of the reversibly reducible material includes, but is not limited to, one or more of the following compounds:

TABLE 1

| Num- | |
|---|---|
| ber | Formula |
| 1 | 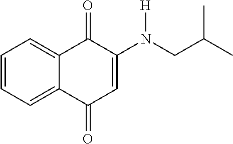 [SMILES: CC(C)CNC1=CC(=O)c2ccccc2C1=O] |
| 2 | 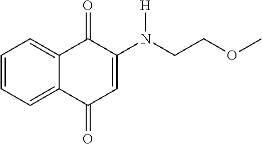 [SMILES: COCCNC1=CC(=O)c2ccccc2C1=O] |
| 3 | 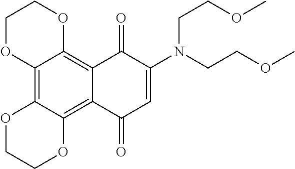 [SMILES: COCCN(CCOC)C1=CC(=O)c2c3c(c4c(c2C1=O)OCCO4)OCCO3] |
| 4 | 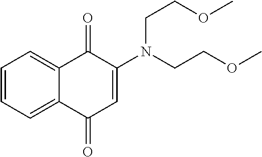 [SMILES: COCCN(CCOC)C1=CC(=O)c2ccccc2C1=O] |
| 5 | 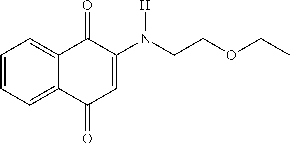 [SMILES: CCOCCNC1=CC(=O)c2ccccc2C1=O] |
| 6 | 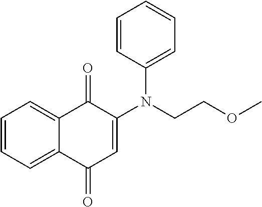 [SMILES: COCCN(C1=CC(=O)c2ccccc2C1=O)c1ccccc1] |
| 7 | 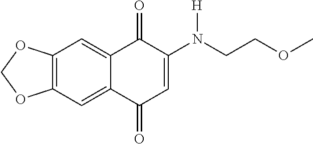 [SMILES: COCCNC1=CC(=O)c2cc3c(cc2C1=O)OCO3] |
| 8 | 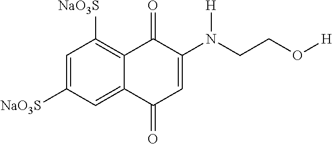 |
| 9 | 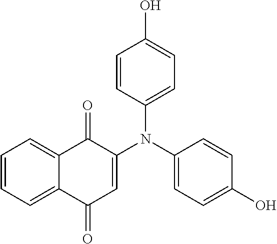 [SMILES: O=C1C=C(N(c2ccc(O)cc2)c2ccc(O)cc2)C(=O)c2ccccc21] |
| 10 | 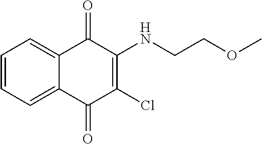 [SMILES: COCCNC1=C(Cl)C(=O)c2ccccc2C1=O] |
| 11 | 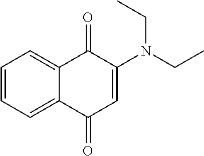 [SMILES: CCN(CC)C1=CC(=O)c2ccccc2C1=O] |
| 12 | 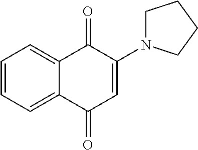 [SMILES: O=C1C=C(N2CCCC2)C(=O)c2ccccc21] |
| 13 | 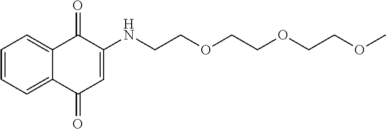 [SMILES: COCCOCCOCCNC1=CC(=O)c2ccccc2C1=O] |
| 14 | 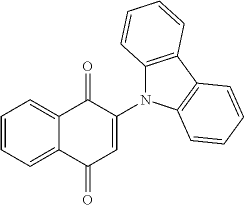 [SMILES: O=C1C=C(n2c3ccccc3c3ccccc32)C(=O)c2ccccc21] |
| 15 | 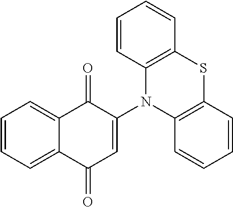 [SMILES: O=C1C=C(N2c3ccccc3Sc3ccccc32)C(=O)c2ccccc21] |
| 16 | 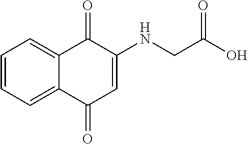 [SMILES: O=C(O)CNC1=CC(=O)c2ccccc2C1=O] |
| 17 | 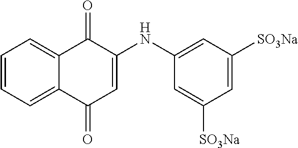 |
| 18 | 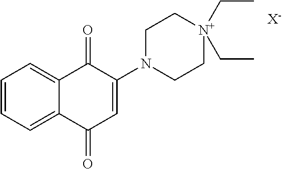 |

In some embodiments, modifying the Z group in formula I above adjusts the solubility of the resulting compound. For example, in one embodiment, compound number 1 has a solubility of 0.1 M in acetonitrile (electrolyte solution), while compound number 2 has a solubility of 0.3 M in acetonitrile. In certain embodiments, adding a second group on the N atom increases the solubility of the compound. For example, compound number 11, where X is CH₂CH₃, has a solubility of 0.7 M in acetonitrile, which is substantially higher than that of compound numbers 1 and 2, where X is H. The second group on the N atom in compound number 11 also provides excellent electrochemistry properties. Additionally or alternatively, in some embodiments, a halogen in the R⁵ position increases the solubility of the compound. For example, inserting Cl at the R⁵ position, as in compound number 10, increases the solubility as compared to compound 2, which is the identical compound without Cl at the R⁵ position.

In some embodiments, the compound includes both an anolyte and a catholyte in the same molecule. For example, in compound numbers 14 and 15, the nitrogen-containing cycles are part of a ring system that is a well-known anolyte material. In certain embodiments, such compounds may form a one-molecule redox flow battery (RFB). In some embodiments, the water-solubilizing functionality may be shifted from the quinone core to the nitrogen side, such as, for example, in compound numbers 16-18. Referring to compound number 13, in some embodiments, the compound is a liquid. In one embodiment, the liquid compound provides a solvent-free version of an RFB. In another embodiment, the solvent-free version of the RFB provides substantially higher power density as compared to RFBs including solvents.

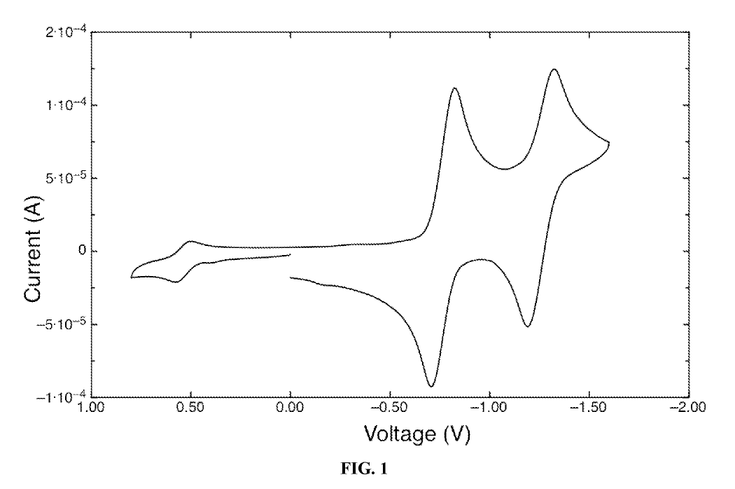

The reversibly reducible material according to one or more of the embodiments disclosed herein is soluble in at least one solvent for redox flow batteries. Accordingly, also provided herein is a negolyte for use in redox-flow batteries, the negolyte including the reversibly reducible material and a redox flow battery solvent. Such solvents include, but are not limited to, acetonitrile, propylene glycol, propylene carbonate, dimethyl acetamide, and dimethyl formamide. For example, the methoxyethyl derivative has shown good solubility in acetonitrile and excellent electrochemical reversibility. In some embodiments, the negolyte also includes an electrolyte. Suitable electrolytes include, but are not limited to, tetraalkylammonium hexafluorophosphate, perchlorate, tetrafluoroborate, triflate, perfluorobutylsulfonate, lithium salts thereof (e.g., lithium hexafluorophosphate or perchlorate), other organic-soluble materials, any other suitable electrolyte, or a combination thereof. Other suitable electrolytes include, but are not limited to, sulfuric acid and/or potassium hydroxide. An example of the electrochemical trace, with internal ferrocene standard in acetonitrile using tetrabutylammonium hexafluorophosphate electrolyte, is shown in FIG. 1. Although discussed above with regard to non-aqueous redox flow batteries, as will be appreciated by those skilled in the art the instant disclosure is not so limited and may include aqueous redox flow batteries as well.

In certain embodiments, the negolyte includes a high concentration of the reversibly reducible material(s) disclosed herein. For example, in one embodiment, the negolyte includes a reversibly reducible material concentration of at least 0.5 M, at least 1.0 M, at least 1.5 M, at least 2.0 M, between 0.5 and 2.0 M, or any combination, sub-combination, range, or sub-range thereof, along with a similar concentration of electrolyte. Additionally or alternatively, in some embodiments, the reversibly reducible material(s) provide stable double reduction without or substantially without significantly increasing molecular weight. The increased charge storage (i.e., double-reduction) and/or increased solubility of the reversibly reducible material(s) disclosed herein increase the electronic capacity of the negolyte. In another embodiment, the negolyte provides an increased range of operating temperature as compared to existing negolytes. For example, solvents such as acetonitrile, which has a melting point of −45° C. and a boiling point of 82° C. may facilitate low-temperature operation, while solvents such as propylene carbonate, which has a melting point of −49° C. and a boiling point of 242° C. may facilitate both low- and high-temperature operation.

Additionally provided herein, is a method of forming a reversibly reducible material comprising reacting a quinone with an amine in an ethereal solvent. In one embodiment, the method comprises reacting a 1,4-naphthoquinone and an amine in a tetrahydrofuran solvent according to the following reaction:

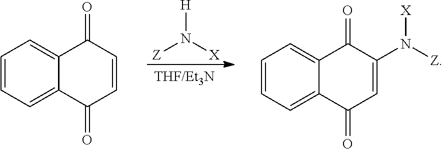

As will be appreciated by those skilled in the art, the solvent is not limited to tetrahydrofuran and may include any other suitable ethereal solvent, such as, but not limited to, dioxane, diethyl ether, glyme, other non-nucleophilic amines such as Hünig's base, or a combination thereof. In certain embodiments, the triethylamine in the reaction above may be replaced by a three-fold excess of amine.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1

For scalable synthesis, reagents must be inexpensive, the reaction high-yielding, and purification methods must be simple and scalable. Many methods reported in the literature are not appropriate for synthesizing the reversibly reducible materials disclosed herein. For example, one method includes reacting 2-bromo 1,4-naphthoquinone with an amine group, as shown in the reaction below:

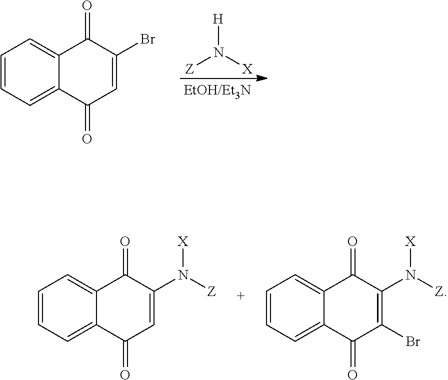

However, when 2-bromo 1,4-naphthoquinone is reacted with an amine group it produces substantial quantities (>15%) of the brominated product, which can be difficult to separate from the desired material. While there are also several literature reports of successful amination of naphthoquinones simply by stirring a suspension of naphthoquinone with the amine in water, none of those experiments could be reproduced by the instant inventors.

Accordingly, the instant inventors developed an efficient route to the desired materials, which, as shown below, includes stirring the quinone and amine (in slight excess) in tetrahydrofuran solvent open to air for 24 to 72 hours.

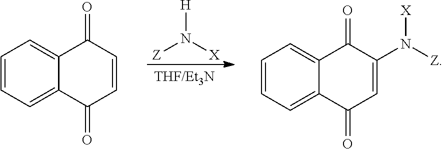

This approach has produced high yields of pure product on scales up to 5 grams, has been demonstrated to work on a wide variety of amines (both primary and secondary), and has yielded materials with minimal need for further purification. Other ethereal solvents, such as dioxane, diethyl ether, or glyme, and other non-nucleophilic amines such as Hünig's base, are also believed to function in this reaction scheme. In cases where the desired amine is inexpensive, a simple 3-fold excess of amine can be utilized in place of added triethylamine.

In more specific examples, following the procedure above using a THF solvent, where X=methoxyethyl and Z=H, the yield was 75%; where X=ethyl and Z=ethyl, the yield was 80%; where X=methoxyethyl and Z=methoxyethyl, the yield was 80%; where X=(CH₂CH₂O)₃CH₃ and Z=H, the yield was 70%. Additionally, for a morpholine adduct, where X and Z are connected through a CH₂CH₂OCH₂CH₂ bridge, the yield was 65%.

### Example 2

Following the formation of reversibly reducible materials as discussed above, various compounds were tested for structure, solubility, and reduction potentials. The results are provided in Table 2 below, with solubility shown in acetonitrile at 23° C. and reduction potentials shown versus ferrocene.

TABLE 2
Solubility and Reduction Potential of Selected Compounds

| | | First | Second |
|---|---|---|---|
| Structure | Solubility | Reduction | Reduction |
| 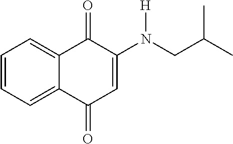 [SMILES: CC(C)CNC1=CC(=O)c2ccccc2C1=O] | 0.1M | −1.266 V | −1.841 V |
| 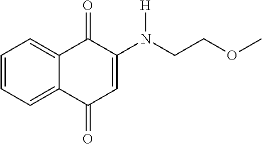 [SMILES: COCCNC1=CC(=O)c2ccccc2C1=O] | 0.3M | −1.316 V | −1.798 V |
| 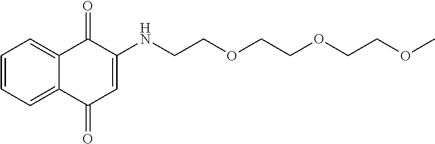 [SMILES: COCCOCCOCCNC1=CC(=O)c2ccccc2C1=O] | Miscible | −1.295 V | −1.784 V |
| 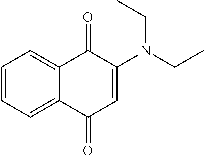 [SMILES: CCN(CC)C1=CC(=O)c2ccccc2C1=O] | 0.7M | −1.362 V | −1.686 V |
| 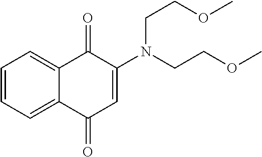 [SMILES: COCCN(CCOC)C1=CC(=O)c2ccccc2C1=O] | Miscible | −1.295 V | 1.667 V |

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A reversibly reducible material comprising the molecular formula: 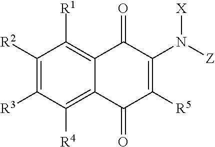
wherein each of R¹, R², R³, and R⁴ are independently selected from the group consisting of hydrogen, oxygen, alkyl, cycloalkyl, O-alkyl, amine, quaternary ammonium, and sulfonate;
wherein R⁵ is selected from the group consisting of hydrogen, halogen, alkyl, alkoxy, and amine;
wherein X is selected from the group consisting of hydrogen, branched or un-branched alkyl chain having 1-8 atoms containing 0-3 oxygen or nitrogen atoms, and substituted or unsubstituted aryl; and
wherein Z is selected from the group consisting of branched or un-branched alkyl chain having 1-8 atoms containing 0-3 oxygen or nitrogen atoms, and substituted or unsubstituted aryl.

2. The material of claim 1, wherein X and Z are connected by an alkyl or heteroalkyl chain containing O, S, or N.

3. The material of claim 1, wherein X, Z, and the N to which they are bound form an aromatic heterocycle selected from the group consisting of a carbazole, phenothiazine, pyrrole, and imidazole.

4. The material of claim 1, wherein R¹, R², R³, and R⁴ comprise an alkyl, O-alkyl, or amine system.

5. The material of claim 4, wherein at least one of R¹ and R², R² and R³, and R³ and R⁴ are linked together to form a ring structure consisting of 3-6 atoms.

6. The material of claim 1, wherein the molecular formula is selected from the group consisting of:

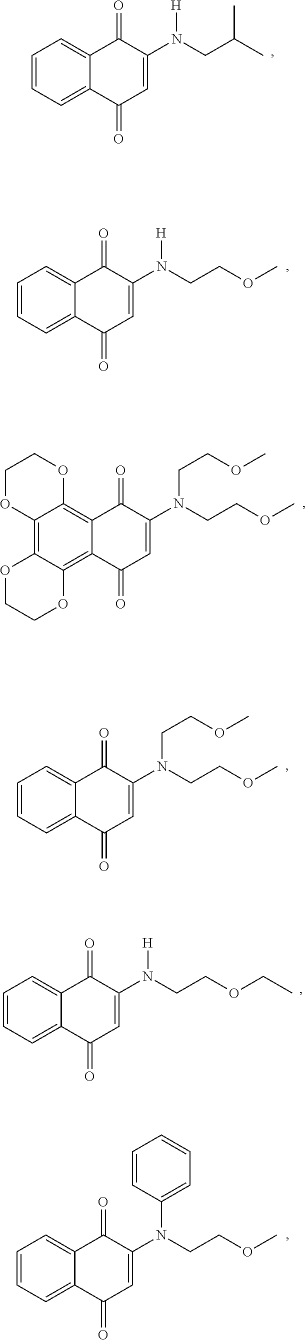 [SMILES: CC(C)CNC1=CC(=O)c2ccccc2C1=O ; COCCNC1=CC(=O)c2ccccc2C1=O ; COCCN(CCOC)C1=CC(=O)c2c3c(c4c(c2C1=O)OCCO4)OCCO3 ; COCCN(CCOC)C1=CC(=O)c2ccccc2C1=O ; CCOCCNC1=CC(=O)c2ccccc2C1=O ; COCCN(C1=CC(=O)c2ccccc2C1=O)c1ccccc1]

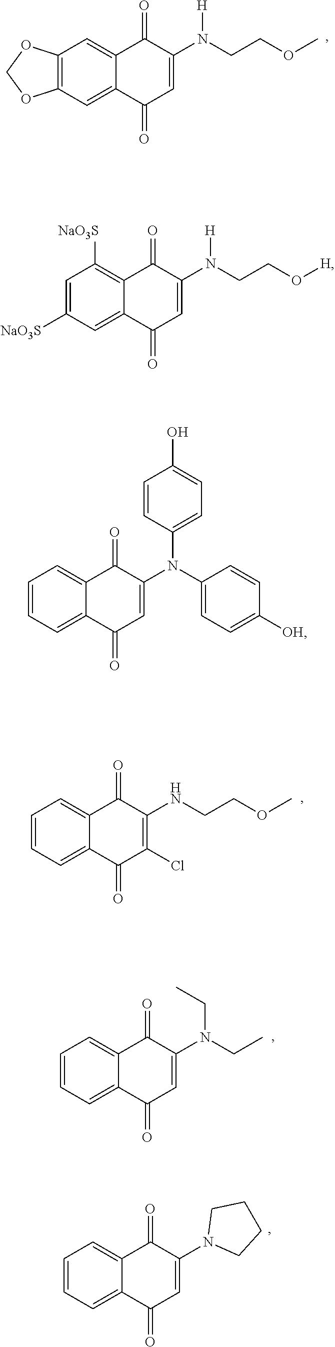

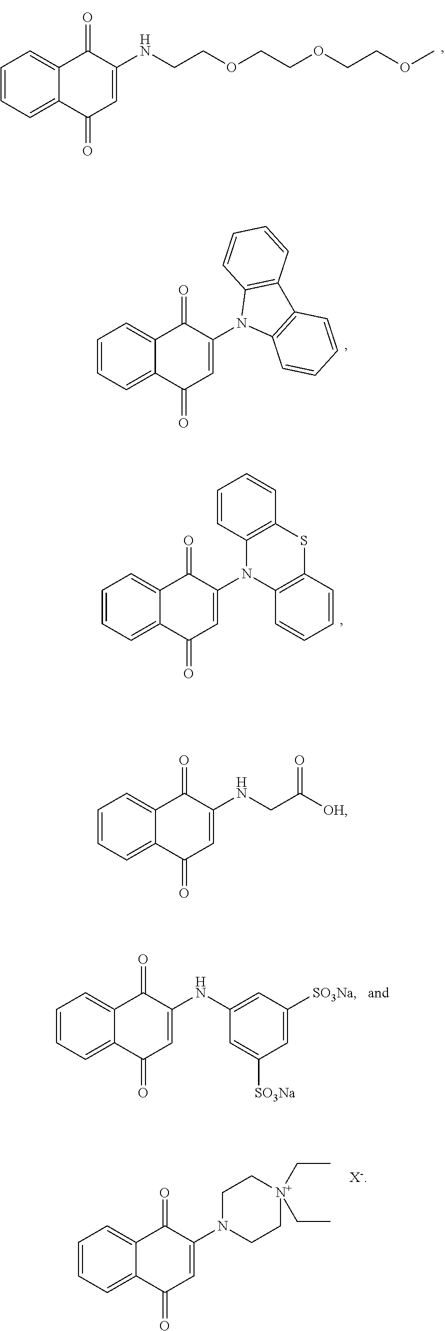

7. A method of forming a reversibly reducible material comprising reacting a quinone with an amine in an ethereal solvent.

8. The method of claim 7, wherein the quinone comprises 1,4-naphthoquinone and the solvent comprises tetrahydrofuran according to the following reaction:

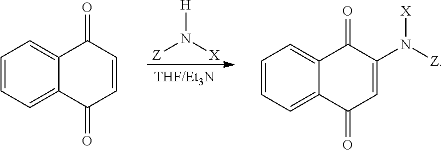

9. The method of claim 7, wherein the ethereal solvent is selected from the group consisting of dioxane, diethyl ether, glyme, and combinations thereof.

10. The method of claim 7, further comprising triethylamine or Hünig's base.

11. The method of claim 7, further comprising a three-fold excess of the amine.

12. A negolyte comprising:
a reversibly reducible material;
a redox flow battery solvent; and
a soluble electrolyte.

13. The negolyte of claim 12, wherein the reversibly reducible material is arranged and disposed to reversibly accept up to two electrons.

14. The negolyte of claim 12, wherein the reversibly reducible material comprises the molecular formula: 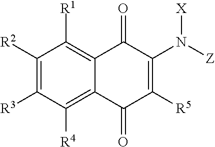
wherein each of R¹, R², R³, and R⁴ are independently selected from the group consisting of hydrogen, oxygen, alkyl, cycloalkyl, O-alkyl, amine, quaternary ammonium, and sulfonate;
wherein R⁵ is selected from the group consisting of hydrogen, halogen, alkyl, alkoxy, and amine;
wherein X is selected from the group consisting of hydrogen, branched or un-branched alkyl chain having 1-8 atoms containing 0-3 oxygen or nitrogen atoms, and substituted or unsubstituted aryl; and
wherein Z is selected from the group consisting of branched or un-branched alkyl chain having 1-8 atoms containing 0-3 oxygen or nitrogen atoms, and substituted or unsubstituted aryl.

15. The negolyte of claim 14, wherein X and Z are connected by an alkyl or heteroalkyl chain containing O, S, or N.

16. The negolyte of claim 14, wherein X, Z, and the N to which they are bound form an aromatic heterocycle selected from the group consisting of a carbazole, phenothiazine, pyrrole, and imidazole.

17. The negolyte of claim 14, wherein R¹, R², R³, and R⁴ comprise an alkyl, O-alkyl, or amine system.

18. The negolyte of claim 17, wherein at least one of R¹ and R², R² and R³, and R³ and R⁴ are linked together to form a ring structure consisting of 3-6 atoms.

19. The negolyte of claim 14, wherein the molecular formula is selected from the group consisting of:

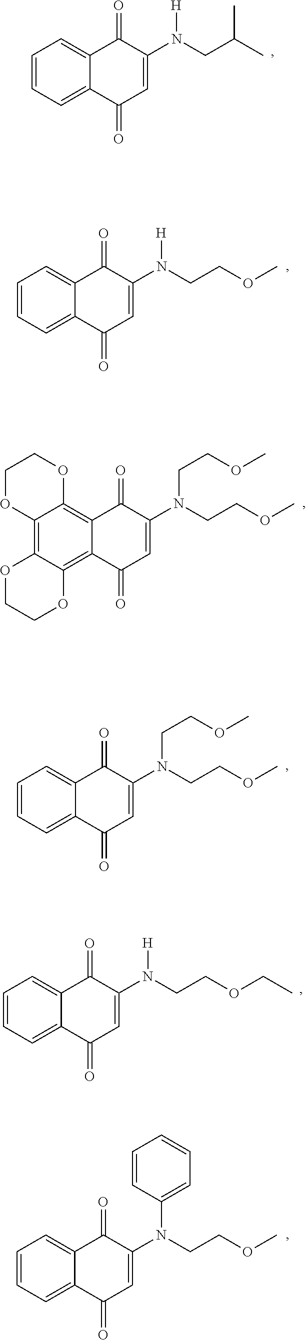 [SMILES: CC(C)CNC1=CC(=O)c2ccccc2C1=O ; COCCNC1=CC(=O)c2ccccc2C1=O ; COCCN(CCOC)C1=CC(=O)c2c3c(c4c(c2C1=O)OCCO4)OCCO3 ; COCCN(CCOC)C1=CC(=O)c2ccccc2C1=O ; CCOCCNC1=CC(=O)c2ccccc2C1=O ; COCCN(C1=CC(=O)c2ccccc2C1=O)c1ccccc1]

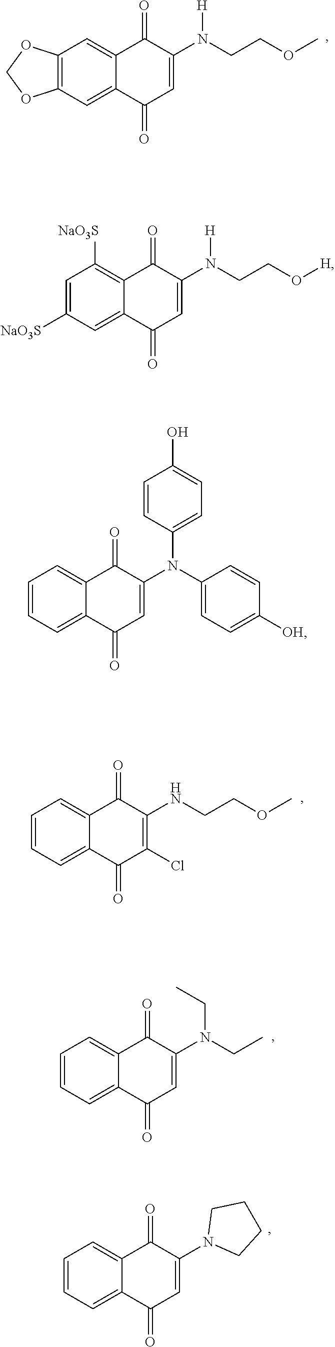

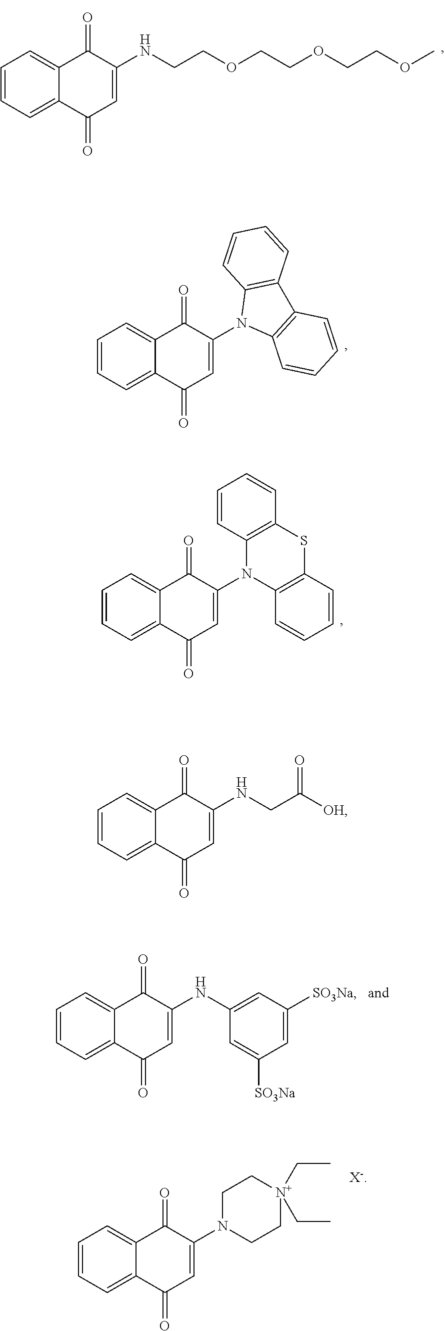

20. The negolyte of claim 12, wherein the solvent is selected from the group consisting of acetonitrile, propylene glycol, propylene carbonate, dimethyl acetamide, and dimethyl formamide; and wherein the electrolyte is selected from the group consisting of tetraalkylammonium hexafluorophosphate, perchlorate, tetrafluoroborate, triflate, perfluorobutylsulfonate, and corresponding lithium salts thereof.
